(19) United States
(12) Patent Application Publication
(10) Pub. No.: US 2018/0361135 A1
(43) Pub. Date: Dec. 20, 2018

(54) Sterile or aseptic connection system

(71) Applicant: Warner Bruce Roberts, Copley, OH (US)

(72) Inventor: Warner Bruce Roberts, Copley, OH (US)

(21) Appl. No.: 15/438,306

(22) Filed: Feb. 21, 2017

Related U.S. Application Data
(60) Provisional application No. 62/297,442, filed on Feb. 19, 2016.

(51) Int. Cl. A61M 39/18; B65D 77/06; A61M 39/24; A61M 39/12; B05B 15/555

(52) U.S. Cl. CPC A61M 39/18; B65D 77/067; F16L 2201/44; A61M 39/12; B05B 15/555; A61M 39/24

(57) ABSTRACT

A method and apparatus to make a sterile or aseptic connection between a container of material, such as a fluid, to a terminus using tubes or hoses interspersed with connectors. The system keeps the material container, material, connectors, tubes or hoses and terminus sterile or aseptic from when making connection through to the evacuation of the material container. The system is substantially comprised of connectors to attach tubes or hoses contiguously by making the connection within an enclosure. The enclosure is used to make and keep the portion surrounding the connectors and enclosure attachments in contact with sterilant or sanitizer as a means to make and keep the system sterile or aseptic. It further includes the means of repeating the process after a material container has been depleted followed by the connecting of a new material container to insure any potential contamination has been eradicated before the process of depletion resumes.

### CROSS-REFERENCE TO RELATED APPLICATIONS

This application claims priority to U.S. Provisional Patent Application No. 62/297,442, which was filed on Feb. 19, 2016

### STATEMENT REGARDING FEDERALLY SPONSORED RESEARCH OR DEVELOPMENT

NA

### REFERENCE TO A “MICROFICHE APPENDIX”

NA

### BACKGROUND OF THE INVENTION

### (1) Field of the Invention

A connection method and apparatus for making a connection, more specifically a method and apparatus for connecting at least a sterile or aseptic tube or hose to provide a sterile or aseptic pathway for material to transit from a material container to a terminus.

### (2) Description of the Related Art

A continuing need exists to prevent contamination of a material, such as a fluid, where a material is needed at a terminus uncontaminated. The devices to be connected would at least be a container holding the material and a terminus. The means of making that connection could comprise of connectors, hoses or tubes and enclosure that lead to a terminus. It provides the ability to make the connection and use for one or more cycles, and provides a method of re-sterilizing or re-sanitizing the system as part of the process of connecting or reconnecting the hoses or tubes after the material container is depleted. Its use would be applicable in a multitude of industries among them medical, bio-medical, food and beverage.

In the medical field, contamination by surface or airborne contaminates of a material that may be injected or ingested by an individual, with a potentially compromised immune system, may be deleterious to the survival of that individual or may be a contributing factor in the continuing length of recovery of the individual. Making a connection, as it exists now, might be removal of a sterile cover on a connector from a material container, swabbing that connector with some form of a sterilizing chemical or in certain instances nothing is done other than making the connection. “Superbugs”, have increased in number and types of antibiotic and sterilant resistant organisms. Numerous studies have shown disinfecting agents are not as effective as the past and only kill a small portion of the contaminant. This is especially the case if the cleansing does not include the entire area and is not robust enough to penetrate below the surface. Since the points of connection are not surrounded entirely or contained during processing with a fluid chemical sterilant, there is no control on adequacy of cleansing and there is not a way to maintain sterility once devices have been processed. By simply having parts of connectors touch a non-sterile item, sterility could be compromised. If the points of contact, at least, or a substantially larger portion of the connecting system were bathed in a sterilant and remained in contact, this potential issue could be overcome. That process could at least include the actions before, during, and after the connection. It could use the sterilant or sanitizer to sterilize or re-sterilize connectors, tubes or hoses, and up to and including the point it reaches a terminus. Depending on the type of terminus other similar means of the system could be employed to make those connections. In some instances, it is advisable and necessary to flush the connectors, hose or tube, and orifice of the terminus. Some systems may make a sterile connection but not flush the pathway.

In the bio-medical field, surface and airborne contamination when making connections is an issue that presents a negative in a different way. A core necessity of bio-medical research is the fidelity of the product that is being utilized. To reduce the potential of contamination, work is often conducted in either sterile glove boxes or a sterile room, where surfaces are cleansed using a multitude of methods, the air is cleaned and positive pressure is utilized to prevent the inflow of micro-organisms that might contaminate. But where basic research is handled as a preliminary step to this higher level of methodology, an errant micro-organism could be introduced, and at minimum, invalidate that portion of the research or at the extreme, provide a non-replicable outcome. Some form of controlling the environment in making connections of tubes or hoses and cleaning of them, could add to the safety and reliability of the operation and its eventual outcome. The system could provide a means of making the connection and keeping the material free from contaminates from source to terminus and might allow for re-charging the system once the source container was depleted. It could replicate the methodology of dean glove boxes or clean rooms by creating an area to isolate the connection process from exposure in an aseptic or sterile environment.

The food as well as the beverage industries have similar issues with contamination that may not be limited to how the connection is made. It may be also who makes the connection, how proficiently, and how accurately to protocol the steps are performed. Other aspects are after the connection is made, are the necessary cleaning steps between changing of the material container carried out on those systems always done and are they done properly to maintain the cleanliness of the system. In food service operations that take as much care as possible, there are a multitude of critical elements that may be disregarded, overlooked, ineffective, or outdated. Increasingly news articles are written concerning some foodborne illness that results in large numbers of people becoming sick and more tragically dying, it may be an issue of micro-organisms becoming resistant to the methods used in cleaning or did a cleansing take place. In highly critical products such as fluids that do not contain preservatives and may only be thermally treated or protected with newer technologies, such as High Pressure Processing, contamination could take place at any point after the materials container has been breached for use. This is may be a concern where material is to be depleted via some type of a tube or hose apparatus that may have points of connection to a device or container. Devices like dispensing systems may be highly susceptible to being a source of illnesses. In a study conducted in 2010 by a leading Food Safety advocacy group, 90 fountain beverages, from both self-service and store-personnel dispensers were tested. 48% of the beverages tested positive for bacteria of multiple types either singly or combinations. In that same time frame the CDC also estimated 76 million cases of foodborne illness occur in the U.S. each year, but only a fraction of these are reported. While the mere presence of bacteria may not be indicative of eventual illness, the potential remains. Contamination may be taking place at three critical points and with store-personnel initiated dispensers, a fourth. The first may be when connection is made to the material container, such as a Bag-in-Box of beverage concentrate. If care is not taken, the two connecting points may become contaminated by store personnel either through direct contact or the connectors touching other surfaces. The second may be the tubing or hose between the BIB and a dispenser or terminus. If not kept clean and sanitized, micro-organism growth or microfilms may propagate. The third may be the exposed dispensing spigot that may come into contact with an individual dispensing the drink. It may either be accidentally or intentionally. With store personnel dispensing the issue may be the same as the last, as well as contacting the inside of the cup. If you could control the environment during connection of the material container by tube or hose with connectors on to terminus as the first step of the dispensing cycle, you would greatly reduce the chance of contaminating the system. Making cleaning of the system part of the change of the material container, you could reduce the potential of a contaminated product and the potential foodborne illnesses.

It is the object of the invention to improve upon systems that allow for making sterile or aseptic connections so that all parts of a system are sterilized or sanitized. It draws upon methods used in isolating procedures such as in sterile glove boxes or clean rooms, and aseptic fillers to sterilize and prevent re-exposing relevant parts of the connecting system to contaminants. It is another objective of the embodiments to provide a sterile or aseptic connection system that is simple and may be substantially modified forms of standard connectors. They may be modified in such ways to allow for the stated operations to be accomplished, such as the addition of a method to connect the enclosure to the connector. The parts may be oriented in ways other than standard practice to make the connection at least more sterile or aseptic. Some parts may have a membrane protecting orifices as a means of protecting that part from contamination before use. Systems parts may be easily replaced by being more accessible. It may be used for a wide variety of needs in segments of industries where sterile or aseptic connections are needed.

It is another objective to provide for sanitation and re-sterilization of the system and its relevant parts to insure any potential contamination has been eradicated before the process of changing the source container.

### BRIEF DESCRIPTION OF THE DRAWINGS

A method and apparatus embodying the invention are hereinafter described, by way of example, referenced to the accompanying drawings.

FIG. 1 shows a view providing substantially at least the elements from embodiment 1

FIG. 2 shows a view providing substantially at least the elements from embodiment 1 before the plurality of the operation has begun

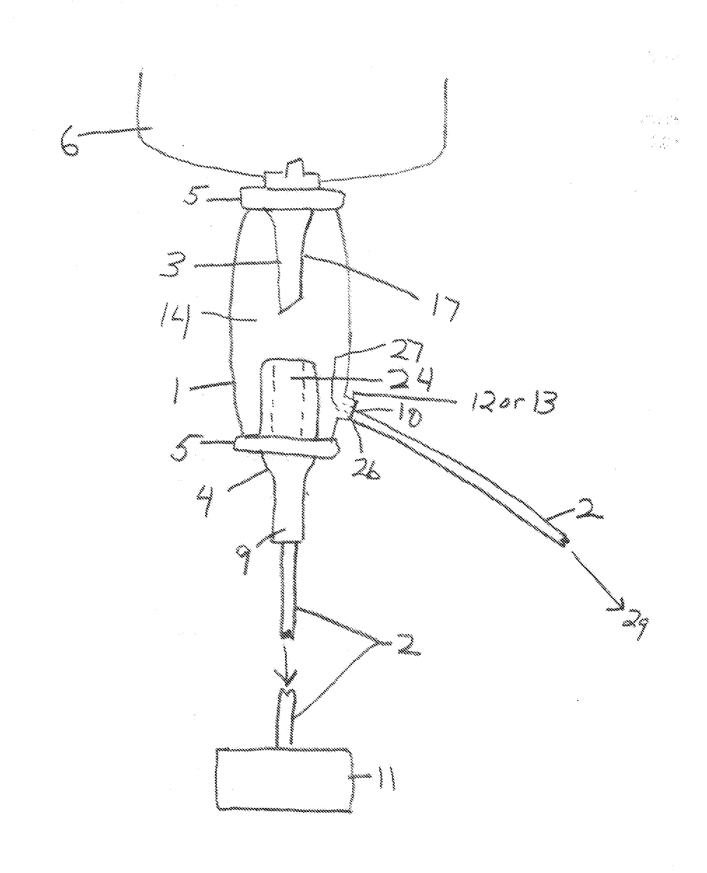

FIG. 3 shows a view providing substantially at least the elements from embodiment 1 after the plurality of the operation is completed.

FIG. 4 shows a view providing substantially at least the elements from embodiment 2.

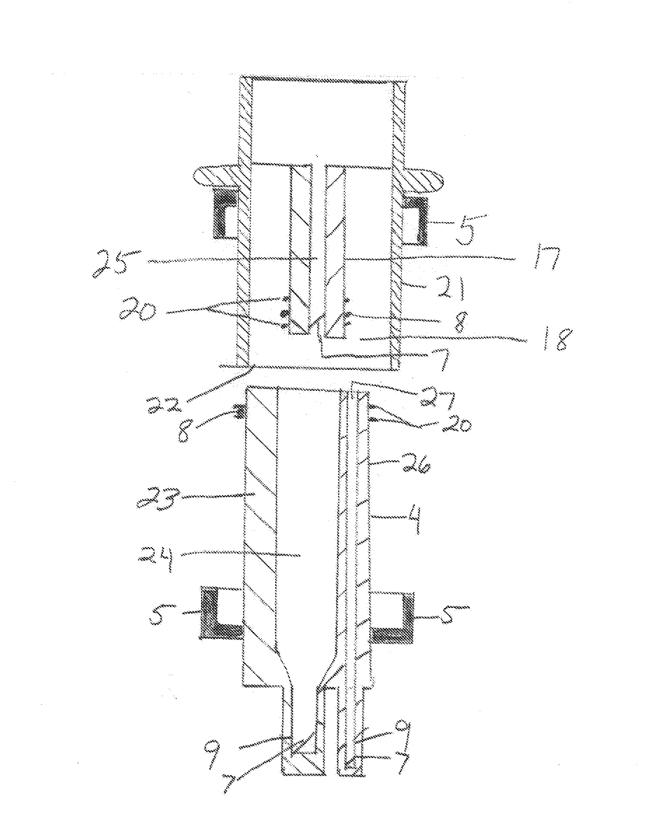

FIG. 5 shows a view providing substantially at least the elements from embodiment 2 before the plurality of the operation has begun.

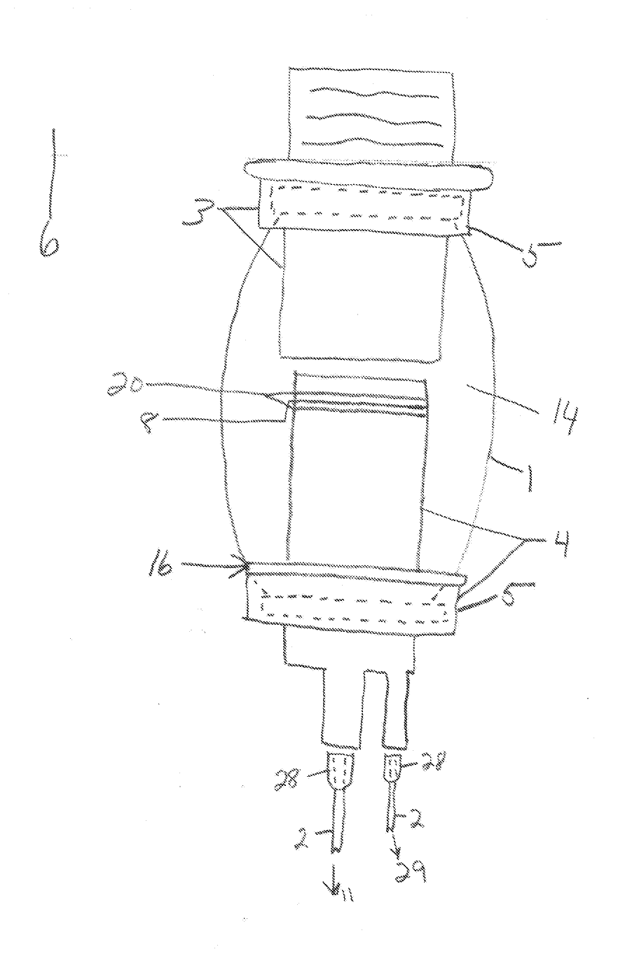

FIG. 6 shows a view providing substantially at least the elements from embodiment 2 after the plurality of the operation is completed.

### DETAILED DESCRIPTION OF EMBODIMENTS

### First Embodiment

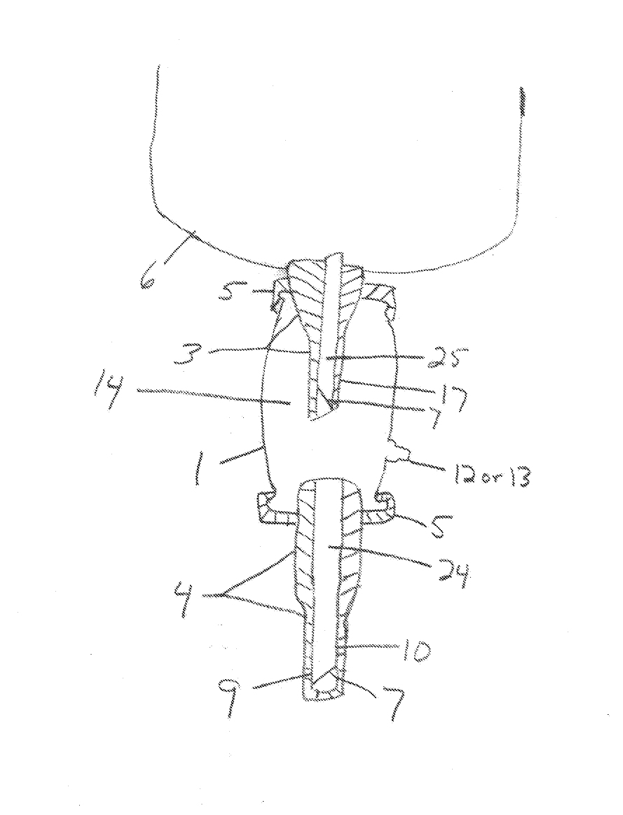

An assembly FIG. 1 that facilitates the process of making a sterile or aseptic connection between material container 6 and terminus 11. The system may prevent contamination of the system during the connection phase through to depletion of material container 6. It may create an entirely enclosed sterile or aseptic field to make the connection within and may subsequently sterilize or sanitize connected hoses or tubes 2. Material flows from material container 6 to terminus 11 without a point which material could become contaminated. System may be comprised of material container 6, a removable and replaceable enclosure 1, hoses or tubes 2, connectors 3, 4, and terminus 11. In this embodiment tubes or hoses 2 and connectors 3, 4 may be of a predetermined size for the transmittal of specialized material to terminus 11 in precise quantities. In this embodiment, connectors 3, 4 may further include enhancements like enclosure attachment 5, to provide means of securing enclosure 1 to connectors to conform to the standards of connection system. Another enhancement could be enclosure 1 may be modified to incorporate alternative sterilant connectors 12 or 13 so it may facilitate the dispersal of sterilant or sanitizer 14 into enclosure 1 by means of a separate hose or tube 2. This may be needed where sterilant or sanitizer port 27 in terminus side connector 4 may not fit due to the relative size of connectors 3, 4.

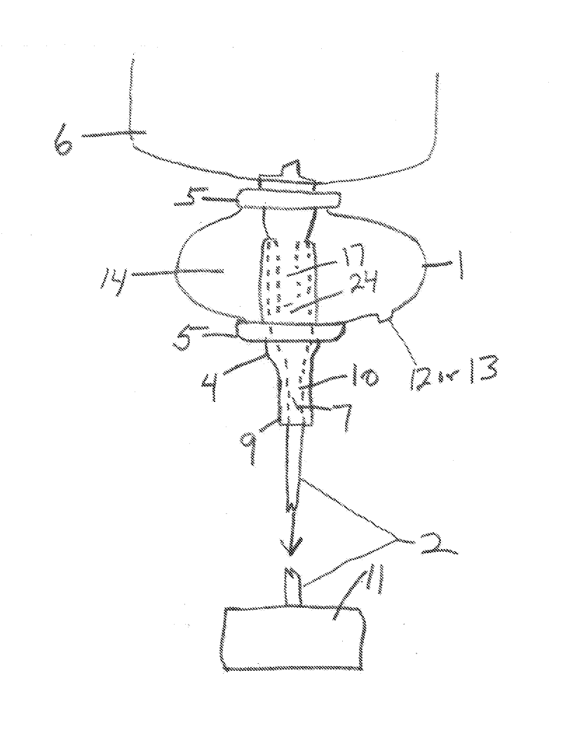

To operate, enclosure 1 is initially attached to terminus side connector 4 by enclosure attachment 5 and may be held in place with securing means 16. While terminus side connector 4 and enclosure 1 is elevated with the orifice that will connect to the material side connector 3 being the elevated portion, sterilant or sanitizer 14 is dispersed gradually into enclosure 1 by means of hose or tube 2 via alternative sterilant connector 12 or 13 from sterilant or sanitizer source 29 propelled by sterilant or sanitizer propulsion mechanism 15. This begins the sterilization of terminus side connector 4 and its parts before continuing the connection process. When sterilant or sanitizer 14 reaches the unattached orifice of enclosure 1, sterilant or sanitizer propulsion mechanism 15 is turned off and enclosure 1 is attached to material side connector 3 using enclosure attachment 5. This begins to allow for the sterilization or sanitization of material side connector 3 and other exposed areas within enclosure 1. Securing means 16 may be used in this step. Sterilant or sanitizer propulsion mechanism 15 is once again started and material propulsion mechanism 19 is started to draw sterilant or sanitizer 14 through female socket 24 and on into hose or tube connection 10 to hose or tube 2 which ends at terminus 11. Once a predetermined amount of time has been completed for this process, sterilant or sanitizer propulsion mechanism 15 and material propulsion mechanism are stopped. Connectors 3, 4 are brought towards each other decreasing the length of enclosure 1 until connection is completed within enclosure 1 FIG. 3. It is maintained in place and may be a completely enclosed system that has been sterilized or sanitized and may prevent further exposure to contaminates. This may insure a sterile product throughout the cycle of evacuating material container 6. Material propulsion mechanism 19 is then run to prime system with material and is halted when material has reached terminus 11 or continues if that is the requirement. If it is halted, it can be activated on demand as part of depletion cycle. Once material container 6 is completely evacuated, the process is reversed and connection process commences again. Enclosure 1 can be replaced as needed to facilitate a continuous operation. Replacing connector 4 would follow the steps of original installation by making connection of hose or tube 2 from sterilant source 29 to enclosure 1 by way of alternative sterilant connectors 12 or 13. To attach hose or tube 2 at terminus 11 to terminus side connector 4 is by way of sleeved connector 9 that is filled with sterilant or sanitizer 14 and hose or tube connector 10. When in an upright position hose or tube connector 10 within sleeved connector 9 is surrounded by sterilant or sanitizer 14. Hose or tube 2 is inserted into sleeved connector 9 while filled with sterilant or sanitizer 14 and on to hose or tube connection 10 until the connection is completed. Cap 28 to maintain sterilant or sanitizer 14 within sleeved connector 9 may be used.

Sterilant propulsion mechanism 15 and material propulsion mechanism 19 may be a peristaltic pump or other types that would prevent contact between the fluid and pump, and it may have variable speeds. Others propulsion forms could be gravity, pistons, pumps, bellows, and pressure.

Material side connector 3 may be attached to material container 6 and might be enhanced by incorporating a check valve 7 to prevent material from being introduced back into material container 6.

Terminus side connector 4 might contain a check valve 7 in hose or tube connection 10 to prevent backflow of material into female socket 24 and maintain flow of material in the direction of terminus 11.

Alternative sterilant or sanitizer connector 12 or 13 may contain check valves 7 to prevent backflow from enclosure 1 into hose or tube 2 leading to sterilant or sanitizer source 29.

Securing means 16 may be a plurality of types and may include screw on, clamp, wire clamp, banjo, and compression clamp.

### Second Embodiment

An assembly FIG. 3 that facilitates the process of making a sterile or aseptic connection between material container 6 and terminus 11. Enclosure 1 may be constructed of an FDA approved food grade material and enclosure 1 having a predetermined shape, cross-sectional shape and length so that it may allow the completion of joining of connectors 3, 4. It has orifices at either end of a predetermined diameter so it easily fits over connectors 3, 4 and enclosure attachment 5. Enclosure attachment 5 maintains enclosure 1 in place surrounding connectors 3, 4 and may use securing means 16. These parts facilitate changing of enclosure 1 when needed as well as to facilitate changing of material container 6.

Material side connector 3 may be a male connector and may reside on material container 6 and may have check valve 7 at its endpoint of nozzle 17 to prevent backflow into material container 6. It may have an O ring 8 fitting into an O ring groove 20 to customize fit and help maintain connection with terminus side connector 4 which may be a female connector when it is inserted into female socket 24. Terminus side connector 4 may have two forms for connecting hoses or tubes to terminus side connector 4 extending from the distal end. A tube or hose 2 leading from sterilant or sanitizer source 29 terminates within sleeved connector 9 and is attached to hose or tube connection 10. Sterilant or sanitizer 14 enters via conduit of sterilant or sanitizer 26 that leads to sterilant or sanitizer dispensing port 27. Sanitizer dispensing port 27 may have a check valve 7 to prevent backflow. Terminus side connector 4 may also have a material conduit 25 from the point at which material leaves the material container 6 to an orifice at distal end of nozzle 17. Material conduit 25 continues on within female socket 24. Material conduit 25 reaches distal portion of terminus side connector 4 and exits through hose or tube connection 10 contained within sleeved connector 9. Hose or tube connection 10 may contain a check valve 7 to prevent backflow to material container 6. Hose or tube connection 10 contained within sleeved connector 9 connects to hose or tube 2 leading to terminus 11. On the outer aspect of female socket 24 of terminus side connector 4 may contain O-ring 8 positioned in O-ring groove 20 at a predetermined point from the proximal end of female socket 24.

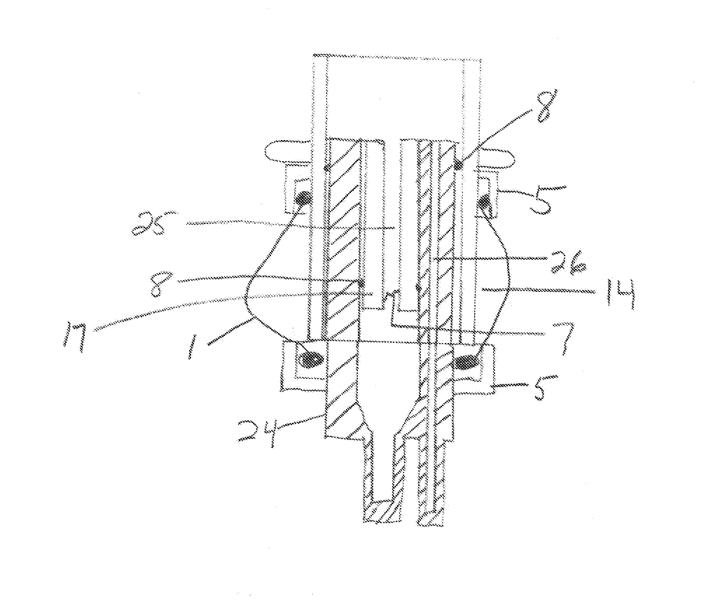

To operate, enclosure 1 is initially attached to terminus side connector 4 by enclosure attachment 5, and may use securing means 16. While terminus side connector 4 and enclosure 1 is elevated with the orifice that attaches to material side connector 3 being the elevated end, sterilant or sanitizer propulsion mechanism 15 is started to propel sterilant or sanitizer 14 from sterilant or sanitizer source 29 then through hose or tube 2 that is connected to hose or tube connection 10 within sleeved connector 9 that leads to sterilant or sanitizer conduit 26 which is then dispersed through sterilant or sanitizer port 27 into enclosure 1. When sterilant or sanitizer 14 reaches orifice of enclosure 1 that attaches to material side connector 3, sterilant or sanitizer propulsion mechanism 15 is stopped. Enclosure 1 is attached to material side connector 3 using enclosure attachment 5 and may use securing means 16. Sterilant or sanitizer propulsion mechanism 15 is started, then material propulsion mechanism 19 is started. Sterilant or sanitizer 14 is drawn into enclosure 1 and then is drawn out through female socket 24 to material conduit 26, then through hose or tube connection 10 that leads to hose or tube 2 that then leads to terminus 11. After a predetermined amount of time both propulsion mechanisms 15, 19 are turned off. Sterilant or sanitizer 14 has then sterilized or sanitized internal portions of a conduit from nozzle 17 to terminus 11. Connectors 3, 4 are brought together decreasing the length of enclosure 1 until the connection within enclosure 1 is completed FIG. 6. It is maintained in place as a completely enclosed system that has been sterilized to prevent exposure to contaminates. This may insure sterile material throughout the cycle of evacuating material container 6. Material propulsion mechanism 19 is then run to prime the system with material and is halted when material reaches terminus 11. Material propulsion mechanism 19 is then available to propel material on demand. When material container 6 is completely evacuated, the processes may be repeated. Enclosure 1 can be replaced as needed. Replacing terminus side connector 4 would follow the steps of the original installation by making the connection of hose or tube 2 from sterilant or sanitizer source 29 to enclosure 1 by way of hose or tube connection 10 within sleeved connector 9. When sleeved connector 9 and hose or tube connection 10 is in an upright position with sterilant or sanitizer surrounding hose or tube connector 10, insertion of hose or tube 2 on to hose or tube connector 10 with connection being completed. The same process would be used to attach terminus side connector 4 by way of sleeved connector 9 and hose or tube connection 10 to terminus hose or tube 2 and then to terminus 11.

Sleeved connector 9 is of a predetermined height to maintain a level of sterilant greater than the height of hose or tube connection 10. Hose or tube connection 10 may contain a check valve 7 at its proximal end to prevent backflow. Cap 28 may be employed to maintain sterilant or sanitizer within sleeved connector 9.

To improve the functionality and safety of the system, source side connector 3 may reside on material container 6 this may remove a potential of contamination of material, as microbiological contaminates cannot be introduced into material container 6 during connection of the material side connector 3. Terminus side connector 4 may reside on terminus 11.

Proprietary connectors may also be designed so system would only work with a specific supplier's material. One means may be use of irregularly placed mortises on a connector and the complimentary tenon on the other connector.

Material propulsion mechanism 19 and sterilant or sanitizer propulsion mechanism 15 may be peristaltic pumps or other forms of pumping that prevent contact between fluid and pump.

A multitude of enclosure attachment 5 and securing means 16 could utilize but not be limited to tongue and groove, clamp, banjo and other tight sealing methods.

Alternative sterilant connectors 12, 13 on enclosure 1 may be utilized, as well as other means of dispersing the sterilant or sanitizer 14 within enclosure 1.

The system could utilize a manual sequence, be an automated or a combination system based on plurality of needs.

What is claimed:

1. A system providing a means to connect and disconnect hoses or tubes that have interposed connectors and may be contained within an enclosure and is comprised of:
(a) An enclosure with associated means of attachment to connectors it that allows for at least the length to be reduced to facilitate joining of connectors within enclosure and also may provide a means to disperse a sterilant or sanitizer within enclosure so that it may make enclosure and what may be surrounded sterile or aseptic.
(b) Sterilant or sanitizer to sterilize or sanitize parts of system that reside within enclosure to be dispersed within enclosure before an actual connection may be made and may remain in continued contact throughout a process of making connection and remain so until there may be a need to disconnect.
(c) A propelling mechanism to propel material and sterilant or sanitizer by means of hose or tube.

2. The system recited in claim 1 may be comprised of an improvement by providing a means of enclosure being replaceable and may be located in an accessible area that may be outside of any apparatus that may have a need for the system.

3. The system recited in claim 1 may be comprised of an improvement by providing a means of enclosure so it may keep all parts within enclosure sterile and isolated for the entirety of pre-connecting through to disconnect, as well may sterilize or sanitize hoses or tubes.

4. The system recited in claim 1 may be an improvement providing the system uses only a sterilant or sanitizer to make the system sterile or aseptic.

5. The system recited in claim 1 may be an improvement providing a person with ordinary skills in relevant art may be able to operate and maintain the system.

6. The system recited in claim 1 may be an improvement providing said attachment of said enclosure to said connectors may be operated manually.

7. The system recited in claim 1 may be an improvement providing a means of being directly connected to material container.

8. The system recited in claim 1 may be an improvement providing source side connector may be a male connector and may reside on material container and terminus side connector may be a female connector.

9. In embodiment 1, enclosure may be modified to incorporate a multitude of alternative connections to hoses or tubes conveying sterilant or sanitizer to facilitate the dispersal of the sterilant or sanitizer into enclosure.

10. In embodiment 2, supply side connector may be a male connector and may be further comprised of a nozzle like projection that contains a check valve at distal end of nozzle and have an O-ring in an O-ring groove to serve as a device to seal and a means of maintaining a connection and be surrounded by an outer collar as a protective covering to protect from potential contamination before a connection process. and may be a mounting point of enclosure attachment

11. In embodiment 2, terminus side connector may be a female connector. and may be further comprised of an outer collar that is also a mounting point of enclosure attachment utilized in securing enclosure to connector.

12. (canceled)
